(19) United States
(12) Patent Application Publication
(10) Pub. No.: US 2004/0149282 A1
(43) Pub. Date: Aug. 5, 2004

(54) Respiratory monitoring systems and methods

(75) Inventor: Randall S. Hickle, Lubbock, TX (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Scott Laboratories, Inc.

(21) Appl. No.: 10/724,870

(22) Filed: Dec. 2, 2003

Related U.S. Application Data
(60) Provisional application No. 60/430,088, filed on Dec. 2, 2002.

(51) Int. Cl. A61M 15/00

(52) U.S. Cl. 128/203.14

(57) ABSTRACT

The present invention includes a respiratory monitor that improves patient safety through the use of highly responsive monitors and displays highly visible to all clinical personnel even in the absence or failure of an alarm display. The display can be positioned so that clinicians do not have to look away from the patient to view the output of the respiratory monitor. The respiratory monitoring system alerts clinicians of potential problems while automatically taking steps to gather additional information and place an integrated drug delivery system in a safe state (e.g., step down or deactivation) in addition to providing a real-time visual indicator of respiratory rate and estimated tidal volume or respiratory effort and effect. Multiple thresholds that trigger corresponding indicators such as color-coded LEDs provide a quantized display of respiratory effort and effect while also providing a certain level of redundancy. The respiratory effort and effect can also be displayed by the intensity of the LEDs. Other arrays of LEDS provide graded levels of alarms.

### CROSS REFERENCE TO RELATED APPLICATIONS

This application claims priority under 35 U.S.C. §119(e) to U.S. Provisional Patent Application No. 60/430,088, “Respiratory Monitoring Systems and Methods,” filed Dec. 2, 2002, which is hereby incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

1. Field of the Invention

The present invention relates, in general, to respiratory monitoring and, more particularly, to respiratory monitoring associated with medical devices.

2. Description of the Related Art

Every year a significant number of patients suffer severe complications or death due to inadequate, improper or inaccurate respiratory monitoring. Unaided by sensors, it is difficult in some critical circumstances, for even the most highly trained clinician to ascertain whether a patient is moving sufficient air or gas for proper alveolar gas exchange. In an attempt to improve patient safety, a number of respiratory monitoring systems have been developed. However, such systems have not fully met the safety needs of patients, particularly in settings such as sedation and analgesia of the conscious and/or spontaneously breathing patient, as evidenced by continuing reports of negative patient episodes due to inadequate, improper or inaccurate respiratory monitoring.

Capnometry systems have been used with some success in assessing the respiration of a patient by evaluating the partial pressure or percent concentration of exhaled carbon dioxide. When using these systems, carbon dioxide production is implicitly correlated to oxygen consumption via the respiratory quotient, which usually has a value of 0.8. Mainstream capnometers consist of a small infrared gas analysis bench that is mounted directly in the patient's respiratory path providing real-time information regarding the CO₂ level in the patient's respiration. However, the sampling cell used by mainstream capnometers is, in general, relatively bulky and heavy. The sample cell of a mainstream capnometer can be in the way when mounted in the respiratory path, e.g., in front of a patient's face. Sidestream capnometers have a pump that continuously aspirates gas samples from the patient's respiratory path, typically at a sampling flow rate of about 200 ml/min, via a sampling tube that carries the sample gas to a gas analysis bench. The finite transport time from the sampling site to the gas analysis bench introduces an undesirable time lag. When a patient stops breathing, the measured and displayed CO₂ level becomes a flat line at zero mm Hg because there are no exhalations containing CO₂. Further, a patient's inhalation generally draws room air (0.003% CO₂) or gas having zero or negligible carbon dioxide concentration such that the inspired CO₂ is for all intents and purposes zero. Thus, it is difficult to instantly know during inspiration whether a patient is simply inhaling or has stopped breathing all together. The need has therefore arisen for a respiratory monitoring system that provides real-time, unambiguous and instantaneous information regarding a patient's respiratory status and phase of respiration.

Many current respiratory monitoring systems require the use of a face mask, where the mask encapsulates the nose and mouth of a patient to create a sealed region. Different designs of such systems utilize different sensors such as temperature sensors, humidity sensors, and flow meters. Many patients may find face masks to be uncomfortable and anxiety inspiring. In addition, many procedures require oral access (e.g., esophogastroduodenoscopy and oral surgery) which makes sealing face masks inapplicable. Also, the continuous fresh gas flow from an anesthesia machine will dilute the CO₂ in the additional deadspace created by the facemask, resulting in artificially low CO₂ levels. On the other hand, existing respiratory monitoring systems without a sealed facemask may not provide respiratory data of sufficient clinical accuracy. The need has therefore arisen for a respiratory monitor that functions independently of a sealed face mask and monitors respiration with sufficient clinical accuracy.

Existing respiratory monitors are generally integrated with alarm systems, where a clinician is alerted to the presence of respiratory compromise by visual and/or audio alarms. In an operating or procedure room environment, where there are multiple alarm sources and auditory and visual stimuli, it may take a while before the attending clinician is able to determine the cause of the alarm and take appropriate action to remedy the situation. In critical circumstances, rapid diagnosis and intervention can prevent morbid complications. The need has therefore arisen for a respiratory monitoring system that simultaneously alerts the attending clinician of a potential problem while automatically taking steps to gather additional information and placing other aspects of a drug delivery system into a safe state.

Existing alarm algorithms or mechanisms generally alert the attending clinician in the event of an alarm condition. In the event of malfunction of the alarm mechanism itself, e.g., failure of the buzzer for an audible alarm or the LED (light emitting diode) for a visual alarm, an alarm will not be generated even though a critical patient condition is present. The lack of an alarm may lull the clinician into a false sense of security, rendering it even more difficult for the clinician to detect the critical patient condition and take timely corrective action. The need has therefore arisen for an alarm and monitoring system that provides real-time monitoring of respiration throughout the duration of a procedure, where a clinician may still be able to readily ascertain whether respiration has been compromised, even in the absence or failure of an alarm mechanism.

False negative alarm conditions may occur with existing respiratory monitoring systems; that is, respiratory compromise may be present while no alarm is generated to alert the clinician of this condition. For example, existing alarms may be set to warn the clinician if a patient does not take a sufficient number of substantial breaths within a pre-determined time window. By taking shallow but frequent breaths, it may be possible for a patient to meet or exceed the fixed and individual alarm threshold for each monitored parameter such that no alarm is generated even though respiration is compromised. The need has therefore arisen for a respiratory monitoring system that provides anthropomorphic, hierarchic and graded alarms based on varying patient conditions, where, for example, one tier of alarms may be correlated to patient conditions that require increased watchfulness and a second tier of alarms may be correlated to more serious patient conditions that require deactivation of drug delivery. An anthropomorphic alarm paradigm is generally less rigid and more context sensitive because it attempts to emulate human behavior, mental processes and experience. The need has further arisen for a respiratory monitoring system that provides a real-time visual indicator of respiratory rate and estimated tidal volume.

### SUMMARY OF THE INVENTION

The present invention satisfies the above needs by providing a respiratory monitor that improves patient safety in the absence of a sealed face mask. The present invention further provides an integrated respiratory monitor with additional patient monitors and drug administration systems, where the integrated system automatically converts the system to a safe state in the event of a significant respiratory compromise. The present invention even further provides a respiratory monitoring system that operates in real time to allow for immediate responses to critical patient episodes. The present invention also provides a respiratory monitoring system that displays real-time information related to a patient's respiratory condition and uses anthropomorphic and safety-biased alarm and intervention paradigms to minimize distracting alarms and time and motion expenditure. The present invention further provides a respiratory monitor integral with an alarm and visual monitoring system that has a high degree of visibility, where a number of attending clinicians can easily monitor real-time information related to a patient's respiratory condition.

### BRIEF DESCRIPTION OF THE DRAWINGS

FIG. 1 illustrates a block diagram depicting one embodiment of a respiratory monitoring system for use with a sedation and analgesia system in accordance with the present invention;

FIG. 2 illustrates a block diagram of a more detailed view of one embodiment of a respiratory monitoring system in accordance with the present invention;

FIG. 3 illustrates one embodiment of a nasal interface in accordance with the present invention;

FIG. 4 illustrates one embodiment of an ear mount in accordance with the present invention;

FIG. 5 illustrates one embodiment of a support band in accordance with the present invention;

FIG. 6 illustrates one embodiment of a method for pressure waveform analysis and segmentation depicting positive pressure thresholds and negative pressure thresholds in accordance with the present invention;

FIG. 7 illustrates one embodiment of an LED display in accordance with the present invention;

FIG. 8 illustrates one embodiment of a method for employing a respiratory monitoring system in accordance with the present invention; and

FIG. 9 illustrates one embodiment of a method for employing a respiratory monitoring system having alarm conditions in accordance with the present invention.

### DETAILED DESCRIPTION OF THE INVENTION

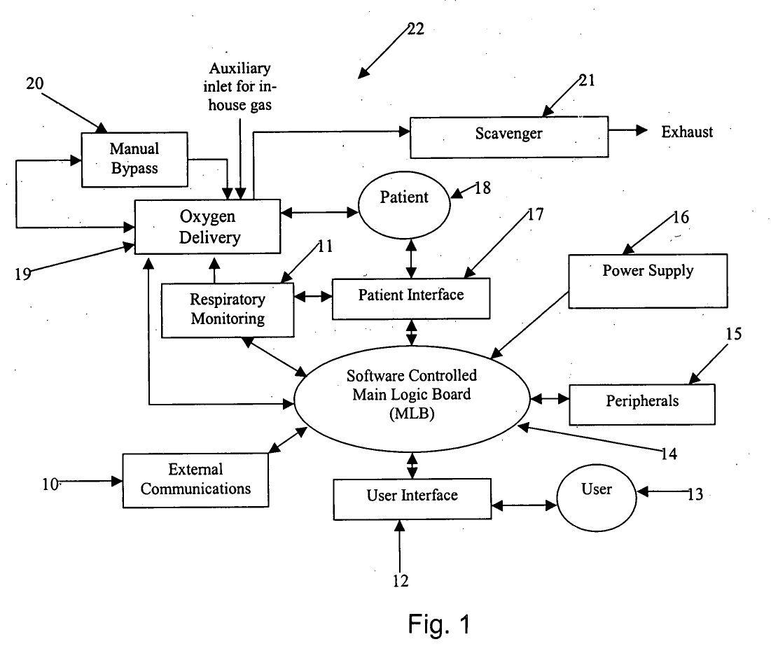

FIG. 1 illustrates a block diagram depicting one embodiment of the present invention comprising a sedation and analgesia system 22 having user interface 12, software controller 14, peripherals 15, power supply 16, external communications 10, respiratory monitoring 11, O₂ delivery 9 with manual bypass 20 and scavenger 21, patient interface 17, and drug delivery 19, where sedation and analgesia system 22 is operated by user 13 in order to provide sedation and/or analgesia to patient 18. Several embodiments of sedation and analgesia system 22 are disclosed and enabled by U.S. patent application Ser. No. 09/324,759, filed Jun. 3, 1999 and incorporated herein by reference in its entirety. It is further contemplated that respiratory monitoring 11 be used in cooperation with sedation and analgesia systems, anesthesia systems and integrated patient monitoring systems, independently, or in other suitable capacities. Embodiments of patient interface 17 are disclosed and enabled by U.S. patent application Ser. No. 09/592,943, filed Jun. 12, 2001 and U.S. patent application Ser. No. 09/878,922 filed Jun. 13, 2001 which are incorporated herein by reference in their entirety.

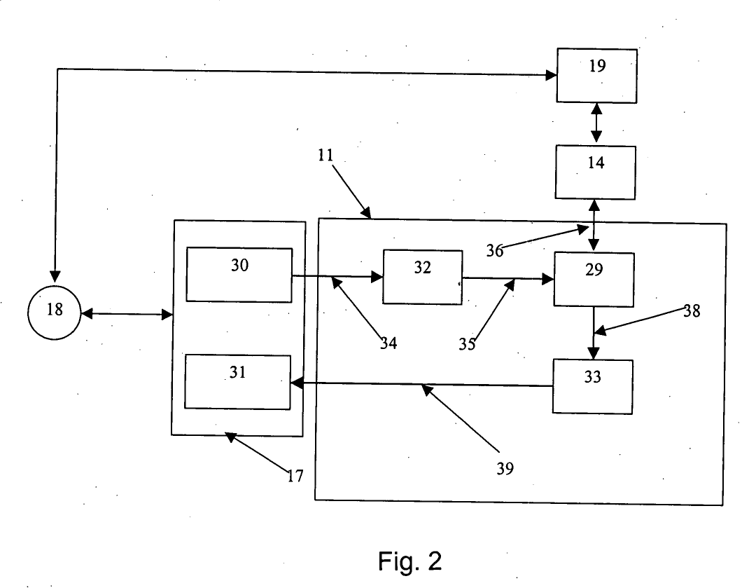

FIG. 2 illustrates a block diagram depicting a more detailed view of one embodiment of respiratory monitoring 11, controller 14, drug delivery 19, and patient interface 17. In one embodiment of the present invention, patient interface 17 comprises nasal cannula 30 and visual display 31. Nasal cannula 30 may deliver oxygen to patient 18, sample the partial pressure or percent concentration of carbon dioxide, and sample nasal pressure associated with inhalation and exhalation. Visual display 31 may be a series of light emitting diodes (LEDs) capable of visually displaying information related to patient respiration. The LEDs may be designed to be reusable with disposable covering lenses. The disposable covering lenses may be designed to amplify the intensity of the LEDs and may also be of shapes (such as arrows or arrowheads) that indicate the direction of gas flow during inhalation and exhalation.

Respiratory monitoring 11 may comprise sensor 32, analog digital input output (ADIO) device 29, and computer programmable logic device (CPLD) 33. Sensor 32 may be a pressure sensor, a humidity sensor, a thermistor, a flow meter, or any other suitable sensor for measuring respiration of patient 18. In one embodiment of the present invention, sensor 32 is a Honeywell DC series differential pressure sensor capable of monitoring from +1 inch to −1 inch of water pressure. The present invention comprises a plurality a sensors that may be associated with individual nares, oral monitoring, both nasal and oral monitoring, intra-vascular monitoring, or other means of employing sensors commonly known in the art.

Still referring to FIG. 2, respiratory monitoring 11 further comprises tubing 34 which interfaces with cannula 30 and sensor 32 in order to measure the pressure variations caused by respiration of patient 18. Tubing 34 may be constructed of any suitable material for providing sensor 32 with accurate pressure measurements from cannula 30 such as, for example, polyvinyl tubing. The characteristics of tubing 34 such as internal diameter, wall thickness and length may be optimized for transmission of the pressure signal. Sensor 32 may output analog signals, where ADIO device 29 converts the analog signals to digital signals before they are transmitted to controller 14 via connection 36. Controller 14 may process the digital signals into respiratory information. Digital signals relating to patient respiration may then be transmitted via connection 38 to CPLD 33, where programming associated with CPLD 33 then controls visual display 31 via connection 39 based on the information contained in the digital signals. In some embodiments of the invention, any of controller 14, ADIO 29, CPLD 33, and sensor 32 may be included or excluded in different combinations or permutations on a single integrated circuit.

In one embodiment of the present invention, controller 14 may control drug delivery 19 based on data received from ADIO device 29, where such data indicates a potentially dangerous patient episode. Controller 14 may be programmed to deactivate drug delivery 19 or reduce drug delivery rate associated with drug delivery 19 in the event of a negative patient episode, or reactivate drug delivery upon receipt of data indicating that patient 18 is no longer experiencing a potentially life-threatening event.

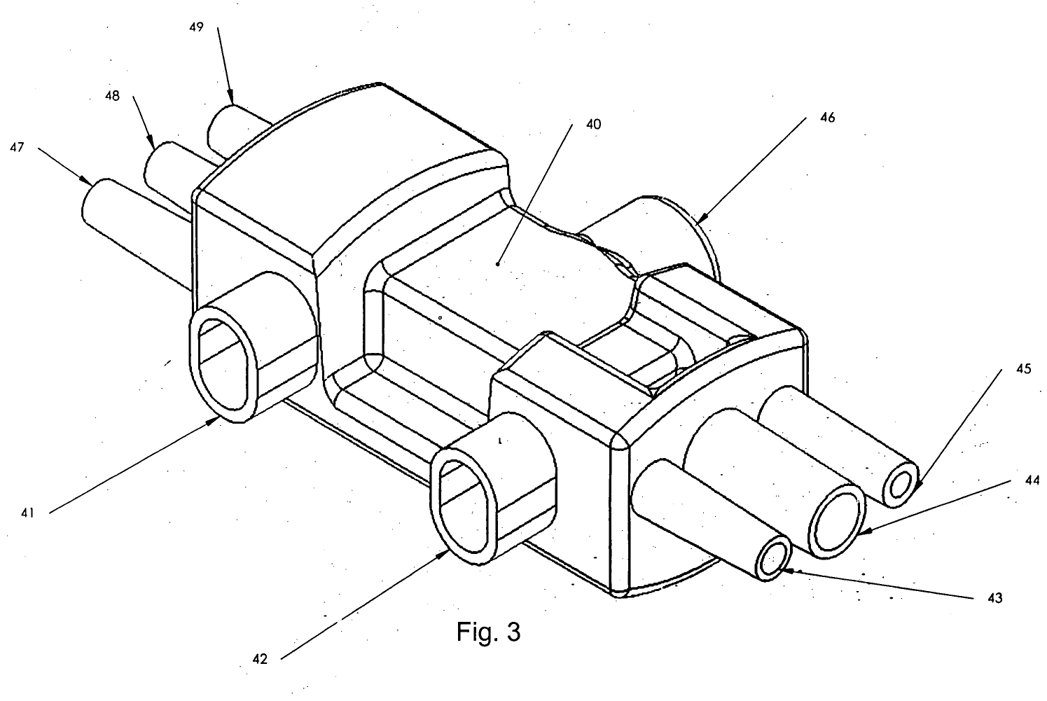

FIG. 3 illustrates one embodiment of nasal interface 40 associated with cannula 30 (FIG. 2). In one embodiment of the present invention, nasal interface 40 comprises first nasal port 41, second nasal port 42, oxygen delivery port 44, first nasal capnography port 48, first pressure sensor port 43, second nasal capnometry port 47, second pressure sensor port 45, oral capnometry port 49, and oral port 46. First nasal port 41 and second nasal port 42 may be designed for placement within or adjacent to the nares of patient 18. An in-house or portable oxygen supply may be connected to oxygen delivery port 44, such that oxygen may be delivered to patient 18 through first nasal port 41 and second nasal port 42 or a grid of ports.

Embodiments of the present invention may comprise monitoring a single nare of patient 18, monitoring multiple nares in the absence of an oral monitor, monitoring patient 18 orally in the absence of nasal monitors, or other suitable monitoring combinations. Oxygen delivery may be optional, orally delivered, nasally delivered, or delivered both orally and nasally. The present invention further comprises a plurality of oxygen delivery ports, where oxygen may be delivered to the nares and/or mouth. It is further consistent with the present invention to deliver a plurality of gases through nasal interface 40 such as, for example, nitrous oxide. A further embodiment of the present invention comprises monitoring a plurality of patient parameters such as, for example, inspired and/or expired oxygen and/or CO₂ concentration or partial pressure via nasal interface 40.

Still referring to FIG. 3, nasal interface 40 may be constructed from nylon, acrylonitrile butadiene styrene (ABS), acrylic, poly-carbonate, or any other suitable material for use in medical devices. It is further consistent with the present invention to monitor CO₂, respiratory rate, respiratory volume, respiratory effort and other patient parameters in the absence of nasal interface 40, where monitoring may be intracorporeal or extracorporeal. The present invention further comprises tubing (not shown) associated with the ports of nasal interface 40, where the tubing may connect nasal interface 40 to a plurality of sensors, gas delivery systems, and/or other suitable peripherals. The tubing may be constructed out of nylon, polyvinyl, silicon, or other suitable materials commonly known in the art.

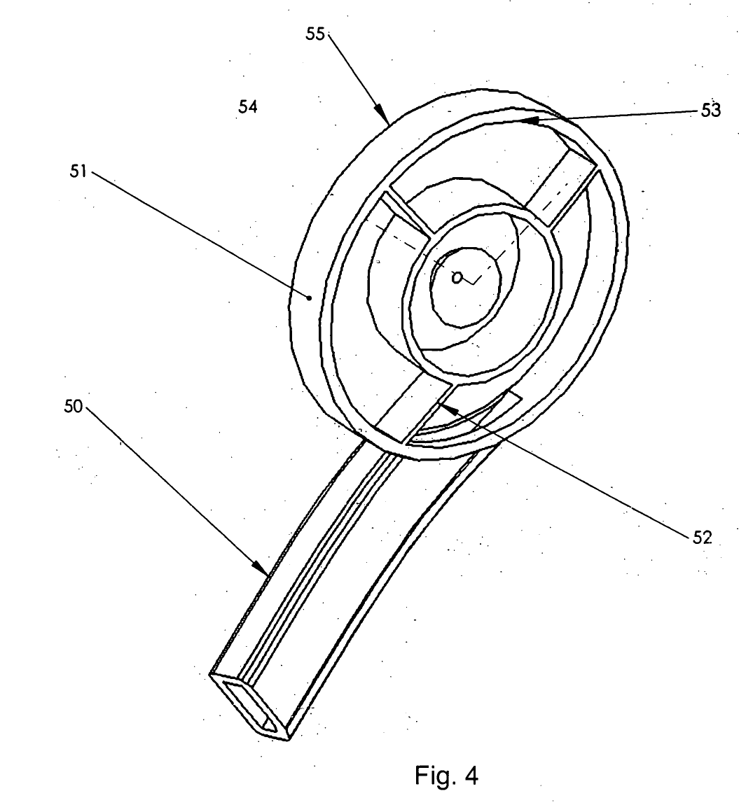

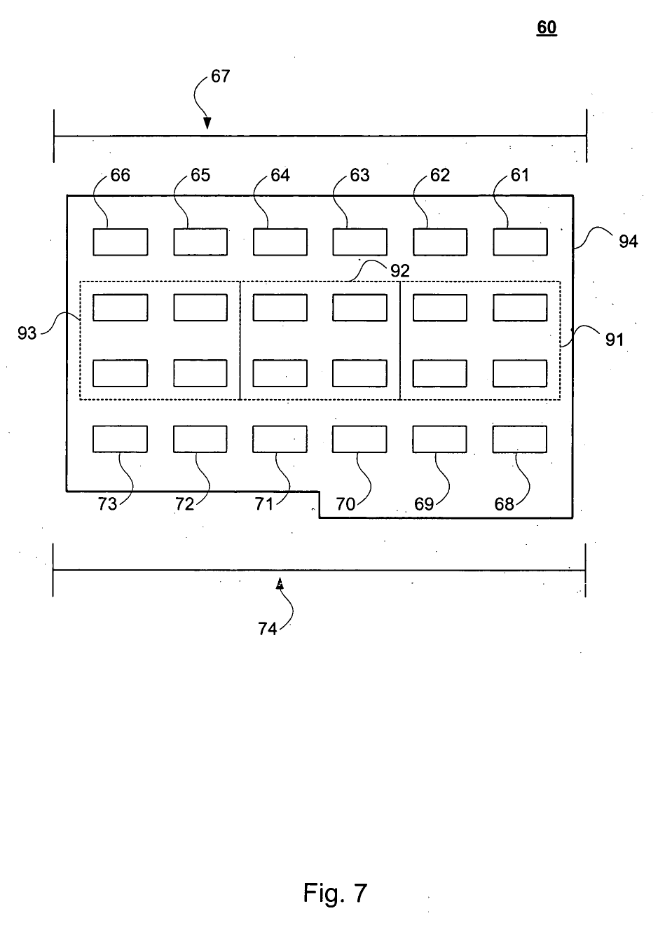

FIG. 4 illustrates one embodiment of ear mount 54 of visual display 31 (FIG. 2). LEDs may be mounted on ear mount 54 which may be adapted for placement on the ear or ears of patient 18. Ear mount 54 comprises stalk 50, base 51, support 52, first interfacing surface 53, and second interfacing surface 55. First interfacing surface 53 may be partially or completely covered in a cushioning surface (not shown), where the cushioning surface is the surface that will come into direct contact with the ear of patient 18. The cushioning surface may be constructed from foam, padded vinyl, or any other material suitable for providing patient comfort. In one embodiment of the present invention, second interfacing surface 55 interfaces with LED display 60 (described below with respect to FIG. 7).

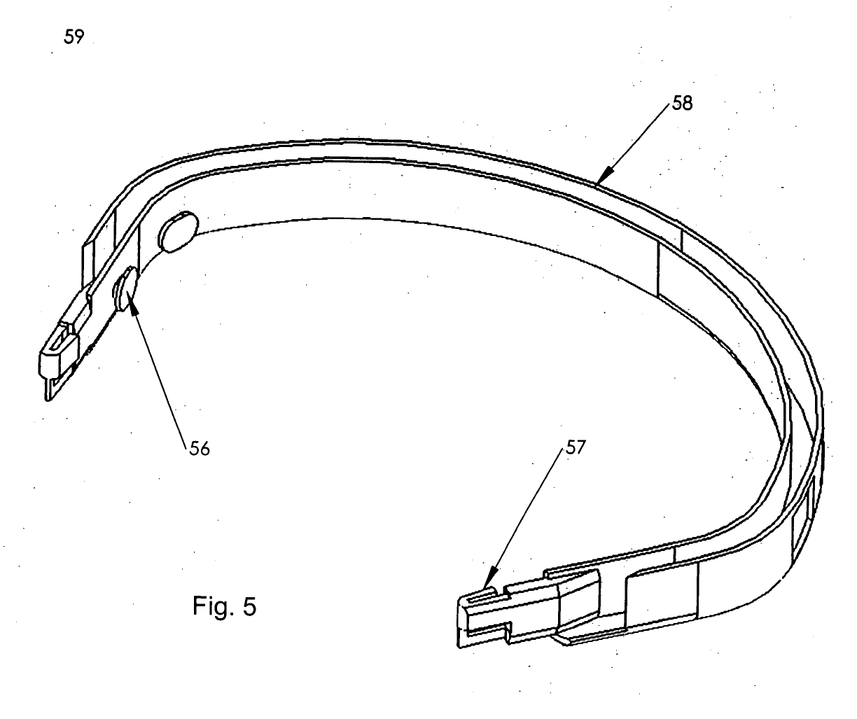

Stalk 50 may be detachably connectable to clasp 57 of support band 58 or permanently affixed to clasp 57 (described below with respect to FIG. 5). Clasp 57 may be a snap fit clasp or any other suitable clasp commonly known in the art. Stalk 50 may be adjustable and/or flexible and/or malleable to provide optimal patient comfort. Ear mount 54 may be constructed from ABS, polycarbonate, or any other suitable material commonly known in the art.

FIG. 5 illustrates one embodiment of support band 59, which comprises support member 58, clasp 57, and comfort band connector 56. Support band 59 may be designed to be detachably removable from ear mount 54 (FIG. 4). Support band 59 may be a head band, where support band 59 is designed to fit snugly around the head of patient 18. Support band 59 may be constructed from any suitable material commonly known in the art, however flexible materials such as, for example, poly-carbonate, silicon, or nylon are preferable. Positioning support band 59, ear mount 54, and LED display 60 (FIG. 7) in the cranial region of patient 18 provides user 13 with a display of high visibility. Support band 59 may be designed to carry a plurality of ear mounts 54 placed on each ear of patient 18. Due to the significant number of procedures requiring patients to lie on their sides, the present invention comprises mounting ear mount 54 over one or both ears. Placing LED display 60 in the cranial region of patient 18 allows user 13 to visually monitor LED display 60 and the respiratory parameters of patient 18 visible to the naked eye simultaneously. The present invention further comprises adapting support band 59 to fit any portion of the body of patient 18, adapting support band 59 for placement on existing medical equipment such as, for example, bed rails, and/or adapting support band 59 to fit on user 13, such as, for example, in the form of a bracelet.

LED display 60 may be utilized in the absence of ear mount 54 and/or support band 59, where LED display 60 is positioned at any suitable location on the body of patient 18, at any suitable location in the operating room, or at any suitable location on the body of user 13. LED display 60 may be integrated with a bracelet, an adhesive for attachment to existing medical structures, or placed in a remote location for remote monitoring. One embodiment of LED display 60 is further disclosed in FIG. 7.

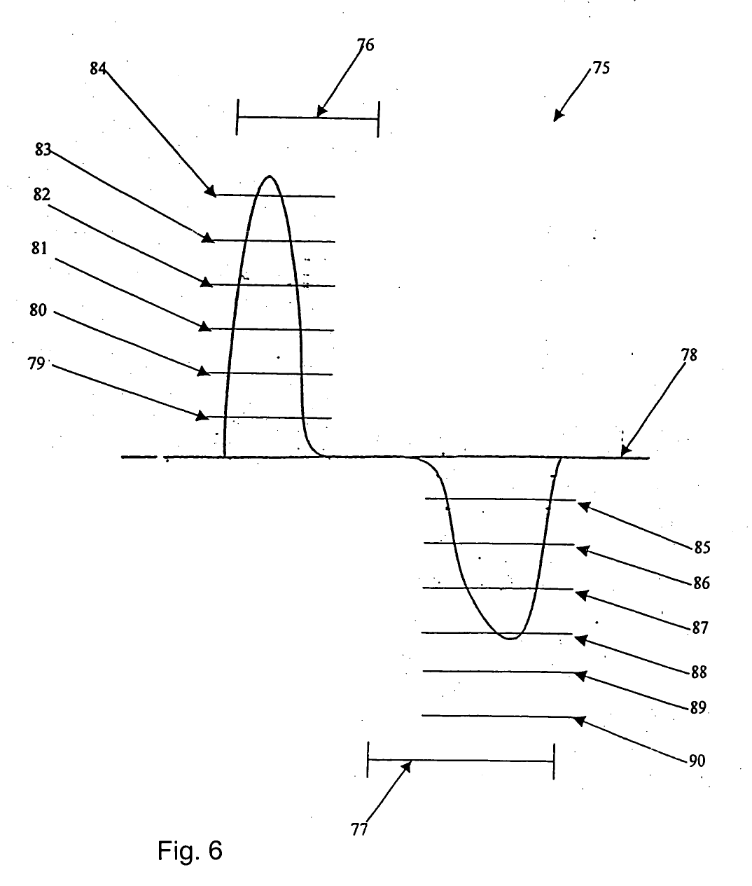

FIG. 6 illustrates one embodiment of a method for pressure waveform analysis and segmentation in accordance with the present invention. Pressure waveform 75 comprises positive pressure region 76, negative pressure region 77, and zero pressure axis 78. FIG. 6 illustrates one full tidal breath of patient 18, where positive pressure region 76 correlates with exhalation and negative pressure region 77 correlates with inhalation. Pressure waveform 75 is at, or close to, the zero pressure axis 78 during the transition from exhalation to inhalation and inhalation to exhalation.

The present invention comprises establishing a series of predetermined positive pressure thresholds 79, 80, 81, 82, 83, 84 and a series of predetermined negative pressure thresholds 85, 86, 87, 88, 89, 90. As patient 18 inhales and exhales, controller 14 will ascertain which of the predetermined thresholds 79, 80, 81, 82, 83, 84, 85, 86, 87, 88, 89, 90 has been exceeded by the respiratory pressure waveform 75. Information relative to magnitude of pressure change associated with inspiration and expiration will then be routed from controller 14 to LED display 60, where specific LEDs associated with corresponding predetermined thresholds will illuminate. Exhalations and inhalations of a low magnitude will result in a minimal number of LEDs lighting, whereas exhalations and inhalations of a high magnitude will result in a greater number of LEDs lighting. By placing LED display 60 in a highly visible area, user 13 or other attending clinicians may visually monitor the respiratory condition of patient 18 in a semi-quantitative manner. Any suitable number of predetermined thresholds 79, 80, 81, 82, 83, 84, 85, 86, 87, 88, 89, 90 may be set at a plurality of pressure levels suitable for a particular patient 18 or application. The present invention further comprises associating positive pressure thresholds 79, 80, 81, 82, 83, 84 with LEDs 61, 62, 63, 64, 65, 66 (FIG. 7), where LEDs 61, 62, 63, 64, 65, 66 are of a particular color such as, for example, blue or gray. The present invention further comprises associating negative pressure thresholds 85, 86, 87, 88, 89, 90 where LEDs 68, 69, 70, 71, 72, 73 are of a particular color different from that associated with exhalation LEDs 67 such as, for example, green. Providing variable color for patient 18 inhalation and exhalation allows user 13 to ascertain at a glance whether patient 18 is inhaling or exhaling, and the pressure magnitude associated with the exhalation or inhalation.

The present invention further comprises establishing alarm parameters within controller 14, where if the inhalations or exhalations of patient 18 do not exceed predetermined pressure thresholds for a predetermined period of time, controller 14 may initiate an alarm condition. In the event of an alarm condition, controller 14 may be programmed to display evidence of the alarm or potentially dangerous patient episode via a series of LEDs 91, 92, 93 associated with LED display 60. For example, first series of LEDs 91 may correlate to a warning condition, second series of LEDs 92 may correlate to a more significant warning condition, and third series of LEDs 93 may correlate to yet a more significant warning condition.

FIG. 7 illustrates one embodiment of LED display 60 in accordance with the present invention comprising first exhalation LED 61, second exhalation LED 62, third exhalation LED 63, fourth exhalation LED 64, fifth exhalation LED 65, and sixth exhalation LED 66, collectively referred to as exhalation LEDs 67. LED display 60 further comprises first inhalation LED 68, second inhalation LED 69, third inhalation LED 70, fourth inhalation LED 71, fifth inhalation LED 72, and sixth inhalation LED 73, collectively referred to as inhalation LEDs 74. LED display 60 further comprises first series of LEDs 91, second series of LEDs 92, third series of LEDs 93, and base 94. In one embodiment of the present invention, base 94 is affixed to ear mount 54, where LEDs associated with LED display 60 face away from patient 18. However, it is contemplated that base 94 be constructed from flexible material or rigid material where base 94 may be placed in any suitable highly visible location.

In one embodiment of the present invention, first exhalation LED 61 corresponds to positive pressure threshold 79, where an exhalation that exceeds first positive pressure threshold 79 will result in first exhalation LED 61 lighting. Second exhalation LED 62 corresponds to second positive pressure threshold 80, where an exhalation that exceeds second positive pressure threshold 80 will result in both first exhalation and second exhalation LEDs 61, 62 lighting. LEDs corresponding to predetermined thresholds will additively light in the above described fashion, where third exhalation LED 63 corresponds to third positive pressure threshold 81, fourth exhalation LED 64 corresponds to fourth positive pressure threshold 82, fifth exhalation LED 65 corresponds to fifth positive pressure threshold 83, and sixth exhalation LED 66 corresponds to sixth positive pressure threshold 84.

The present invention further comprises providing inhalation LEDs 74 where first inhalation LED 68 corresponds to negative pressure threshold 85, where an inhalation that exceeds first negative pressure threshold 85 will result in first inhalation LED 68 lighting. Second inhalation LED 69 corresponds to second negative pressure threshold 86, where an inhalation that exceeds second negative pressure threshold 86 will result in both first inhalation and second inhalation LEDs 68, 69 lighting. LEDs corresponding to predetermined thresholds will additively light in the above described fashion, where third inhalation LED 70 corresponds to third negative pressure threshold 87, fourth inhalation LED 71 corresponds to fourth negative pressure threshold 88, fifth inhalation LED 72 corresponds to fifth negative pressure threshold 89, and sixth inhalation LED 73 corresponds to sixth negative pressure threshold 90.

The thresholds 79-90 may be absolute or relative values. For example, for a pressure sensor where 0 output voltage represents zero or ambient pressure, each threshold may be fixed at a set voltage representing a given pressure level. With a bi-polar, linear pressure sensor where each inch of water pressure is 10 volts of output voltage and 0 V represents ambient (zero) pressure, a first threshold may be set at +0.1 V representing a pressure threshold of 0.01″ of water. However if the zero output voltage drifts on the pressure sensor (“zero drift”), the absolute voltage thresholds will no longer correspond to the desired pressure thresholds. Thus, a preferred embodiment uses relative pressure thresholds whereby the unique voltage corresponding to each threshold is re-adjusted to maintain the desired difference relative to the new output voltage at ambient pressure, in the event of zero drift. This method requires frequent zero calibration of the pressure sensor by exposing it intermittently and briefly to ambient pressure and recording the actual output voltage at zero or ambient pressure.

LED display 60 further comprises first series of LEDs 91, where first series of LEDs 91 may be associated with a first alarm condition; second series of LEDs 92, where second series of LEDs 92 may be associated with a second alarm condition; and third series of LEDs 93, where third series of LEDs 93 may be associated with a third alarm condition. First, second, and third series of LEDs 91, 92, 93 may employ any suitable number of LEDs such as, for example, four LEDs in each series, where the LEDs may be of any suitable color and may be programmed to blink, revolve, or indicate an alarm to user 13 by any other means commonly known in the art. The present invention further comprises employing one or a plurality of illumination devices in cooperation with or in place of LEDs associated with LED display 60 such as, for example, lamps or liquid crystal displays (LCDs). The LEDs associated with the present invention may be configured in a plurality of ways in accordance with the present invention such as, for example, a circular or sinusoidal pattern. Any suitable number of LEDs with corresponding pressure thresholds may be established in accordance with the present invention. Though sensor 32 is a pressure sensor in one embodiment of the present invention, it is contemplated that sensor 32 may be any suitable sensor such as, for example, a temperature sensor, where a waveform may be established corresponding to that sensor, where predetermined thresholds may be established based on the particular characteristics and unique properties of different sensors. It is further contemplated that exhalation LEDs 67 and/or inhalation LEDs 74 grow brighter as the magnitude of exhalation and/or inhalation pressure increases. In one embodiment of the present invention, the increased brightness is accomplished by pulse width modulation of the current or voltage waveform supplied to the LEDs associated with visual display 31.

Providing highly visible LEDs corresponding to the respiratory condition of patient 18 provides user 13 with easily viewable, semi-quantitative respiratory information. The present invention allows user 13 to quickly ascertain at a glance whether patient 18 is inhaling or exhaling, at what rate patient 18 is inhaling and exhaling, and the magnitude of inhalation and exhalation. LEDs associated with a critical patient episode may also be present, alerting attending clinicians in a highly visible manner of a potential problem. Integrating drug delivery 19 with respiratory monitoring 11 provides for the immediate deactivation or stepping down of drug delivery rate in the event of a negative patient episode, whereas it may have taken a while for a clinician to diagnose and respond to the alarm. The series 67 and 74 of LEDs (FIG. 7) provide a quantized visual indicator of the respiratory effect (pressure swings at the airway). In general, a respiratory monitor of effect (the result of a breath such as pressure swings at the airway or exhaled humidity) is more reliable than a monitor of respiratory effort (such as a transthoracic impedance plethysmography) because the latter is fooled when there is an effort but no effect such as in the case of a blocked airway.

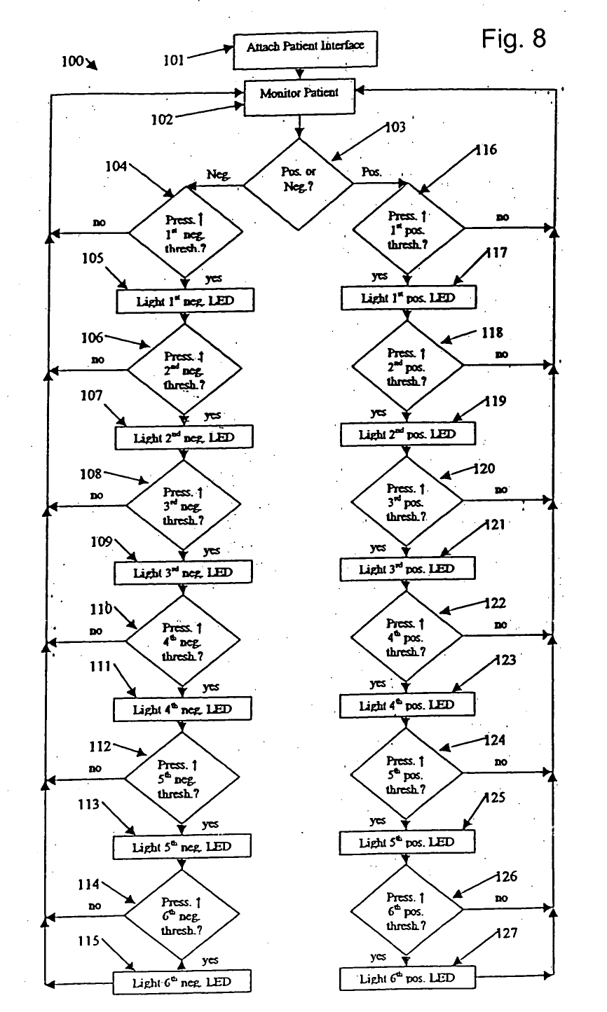

FIG. 8 illustrates one embodiment of method 100 for implementing respiratory monitoring 11 in accordance with the present invention. Method 100 comprises step 101 of attaching the patient interface, comprising fitting patient 18 with visual display 31 and nasal cannula 30. Visual display 31 may be placed at any suitable position on patient 18, on the user, in the operating room, or in a remote location. Nasal cannula 30 may be an integrated oxygen delivery and patient monitoring system, or may be any other suitable means of monitoring the respiratory condition of patient 18. Once visual display 31 and nasal cannula 30 have been properly fitted, method 100 transitions to step 102 of monitoring the patient.

Step 102 of monitoring the patient comprises, in one embodiment of the present invention, integrating respiratory monitoring 11 with patient interface 17, where pressure variations caused by respiration pass from nasal cannula 30 to sensor 32. Step 102 of monitoring the patient may further comprise a plurality of sensors 32, such as thermistors, flow meters, humidity sensors, and/or other sensors commonly known in the art, in cooperation with, or in the absence of a pressure sensor. Signals related to respiratory pressure associated with inhalation and exhalation of patient 18 may be routed to controller 14, where controller 14 is programmed to evaluate the data, output data related to respiratory condition and determine if a negative patient episode has occurred. Alarm conditions associated with respiratory monitoring 11 will be further discussed herein.

Following step 102 of monitoring the patient, method 100 proceeds to query whether pressure evaluated by sensor 32 is a negative pressure or positive pressure, herein referred to as query 103. Negative or sub-ambient pressure is associated with inhalation, whereas positive or supra-ambient pressure is associated with exhalation. Controller 14 comprises programming designed to interpret the signals from sensor 32 as corresponding to either positive or negative pressure. If controller 14 determines that patient 18 is generating negative pressure corresponding to an inhalation, method 100 transitions to query 104 to determine whether the negative pressure exceeds negative pressure threshold 85.

Query 104 comprises controller 14 evaluating signals from sensor 32 to determine if the negative pressure exceeds the predetermined threshold. The predetermined threshold may be set at any pressure suitable for patient 18 or the application at hand. If the negative pressure of inhalation of patient 18 does not exceed negative pressure threshold 85, no LEDs will light on visual display 31, and method 100 will transition to step 102 of monitoring the patient. In further embodiments of the present invention, as will be discussed herein, failing to exceed the predetermined thresholds may result in one or a plurality of alarm responses.

If the negative pressure of the inhalation exceeds negative pressure threshold 85, method 100 proceeds to step 105 of lighting the first negative pressure LED 68. Following step 105 of lighting the first negative pressure LED, method 100 proceeds to query whether the negative pressure associated with the inhalation of patient 18 exceeds the second negative pressure threshold, herein referred to as query 106.

Query 106 comprises programming controller 14 with a second predetermined negative pressure threshold such as, for example, negative pressure threshold 86. Controller 14 will then interpret signals from sensor 32 to determine if the negative pressure associated with exhalation exceeds the negative pressure threshold 86. If the negative pressure does not exceed negative pressure threshold 86, method 100 returns to step 102 of monitoring the patient.

If the negative pressure exceeds negative pressure threshold 86, method 100 proceeds to step 107 of lighting the second negative pressure LED 69. In one embodiment of the present invention, negative pressure of sufficient magnitude to cross negative pressure threshold 86 results in both first inhalation LED 68 and second inhalation LED 69 being illuminated simultaneously. A further embodiment of the present invention comprises pulse width modulation (PWM) of the electrical supply delivered to an LED array. As a greater number of predetermined thresholds are crossed, the pulse width is increased resulting in brighter light intensity of the LEDs. For example, second inhalation LED 69 may have a longer pulse width than first inhalation LED 68, resulting in second inhalation LED 69 having a brighter appearance than first inhalation LED 68. Providing LEDs and multiple pulse width modulations may result in highly visually discernable levels of patient respiration.

Following step 107 of lighting the second pressure LED, method 100 proceeds to query whether the negative pressure associated with patient inhalation exceeds negative pressure threshold 87, herein referred to as query 108. If the negative pressure does not exceed negative pressure threshold 87, method 100 returns to step 102 of monitoring the patient. If the negative pressure exceeds negative pressure threshold 87, method 100 proceeds to step 109 of lighting the third negative pressure LED 70.

Following step 109 of lighting the third pressure LED 70, method 100 proceeds to query whether the negative pressure associated with inhalation exceeds negative pressure threshold 88, herein referred to as query 110. If the negative pressure does not exceed negative pressure threshold 88, method 100 returns to step 102 of monitoring the patient. If the negative pressure exceeds negative pressure threshold 88, method 100 proceeds to step 111 of lighting the fourth negative pressure LED 71.

Following step 111 of lighting the fourth negative pressure LED 71, method 100 proceeds to query whether the negative pressure associated with inhalation exceeds negative pressure threshold 89, herein referred to as query 112. If the negative pressure does not exceed negative pressure threshold 89, method 100 returns to step 102 of monitoring the patient. If the negative pressure exceeds negative pressure threshold 89, method 100 proceeds to step 113 of lighting the fifth negative pressure LED 72.

Following step 113 of lighting the fifth pressure LED 72, method 100 proceeds to query whether the negative pressure associated with inhalation exceeds negative pressure threshold 90, herein referred to as query 114. If the negative pressure does not exceed negative pressure threshold 90, method 100 returns to step 102 of monitoring the patient. If the negative pressure exceeds negative pressure threshold 90, method 100 proceeds to step 115 of lighting the sixth negative pressure LED 73.

The present invention further comprises lighting up all LEDs associated with crossed negative pressure thresholds simultaneously where, for example, if the sixth negative LED 73 is on, all of the LEDs associated with lesser negative thresholds are also illuminated.

Returning to query 103, if controller 14 determines patient 18 is generating positive or supra-ambient pressure corresponding to an exhalation, method 100 transitions to query 116 to determine whether the positive pressure exceeds positive pressure threshold 79.

Query 116 comprises controller 14 evaluating signals from sensor 32 to determine if the positive pressure exceeds predetermined threshold 79. The predetermined threshold may be set at any pressure suitable for patient 18 or the application at hand. If the positive pressure of exhalation of patient 18 does not exceed positive pressure threshold 79, no LEDs will light on visual display 31, and method 100 will continue with step 102 of monitoring the patient. In further embodiments of the present invention, as will be discussed herein, failing to exceed the predetermined thresholds may result in one or a plurality of alarm responses.

If the positive pressure of the exhalation of patient 18 exceeds positive pressure threshold 79, method 100 proceeds to step 117 of lighting the first positive pressure LED 61. Following step 117 of lighting the first positive pressure LED, method 100 proceeds to query whether the positive pressure associated with exhalation exceeds the second positive pressure threshold 80, herein referred to as query 118.

Query 118 comprises controller 14 interpreting signals from sensor 32 to determine if the positive pressure associated with exhalation exceeds the positive pressure threshold 80. If the positive pressure does not exceed positive pressure threshold 80, method 100 returns to step 102 of monitoring the patient.

If the positive pressure exceeds positive pressure threshold 80, method 100 proceeds to step 119 of lighting the second positive pressure LED 62. In one embodiment of the present invention, positive pressure of sufficient magnitude to cross positive pressure threshold 80 results in both first exhalation LED 61 and second exhalation LED 62 being illuminated simultaneously. A further embodiment of the present invention comprises pulse width modulations (PWM) of the electrical supply to an LED array. As a greater number of predetermined thresholds are crossed, the pulse width is increased, resulting in an increase in the light intensity of the LEDs. For example, second exhalation LED 62 may have a longer pulse width than first exhalation LED 61, resulting in second exhalation LED 62 having a brighter appearance than first exhalation LED 61. Providing LEDs and multiple pulse width modulations may result in highly visually discernable levels of respiration.

Following step 119 of lighting the second pressure LED 62, method 100 proceeds to query whether the positive pressure associated with exhalation exceeds positive pressure threshold 81, herein referred to as query 120. If the positive pressure does not exceed positive pressure threshold 81, method 100 returns to step 102 of monitoring the patient. If the positive pressure exceeds positive pressure threshold 81, method 100 proceeds to step 121 of lighting the third positive pressure LED 63.

Following step 121 of lighting the third positive pressure LED 63, method 100 proceeds to query whether the positive pressure associated with exhalation exceeds positive pressure threshold 82, herein referred to as query 122. If the positive pressure does not exceed positive pressure threshold 82, method 100 returns to step 102 of monitoring the patient. If the positive pressure exceeds positive pressure threshold 82, method 100 proceeds to step 123 of lighting the fourth positive pressure LED 64.

Following step 123 of lighting the fourth positive pressure LED 64, method 100 proceeds to query whether the positive pressure associated with exhalation exceeds positive pressure threshold 83, herein referred to as query 124. If the positive pressure does not exceed positive pressure threshold 83, method 100 returns to step 102 of monitoring the patient. If the positive pressure exceeds positive pressure threshold 83, method 100 proceeds to step 125 of lighting the fifth positive pressure LED 65.

Following step 125 of lighting the fifth positive pressure LED 65, method 100 proceeds to query whether the positive pressure associated with exhalation exceeds positive pressure threshold 84, herein referred to as query 126. If the positive pressure does not exceed positive pressure threshold 84, method 100 returns to step 102 of monitoring the patient. If the positive pressure exceeds positive pressure threshold 84, method 100 proceeds to step 127 of lighting the sixth positive pressure LED 66.

The present invention further comprises lighting up all LEDs associated with crossed positive pressure thresholds simultaneously where, for example, if the LED 66 is on, all of the LEDs associated with lesser positive thresholds are also illuminated.

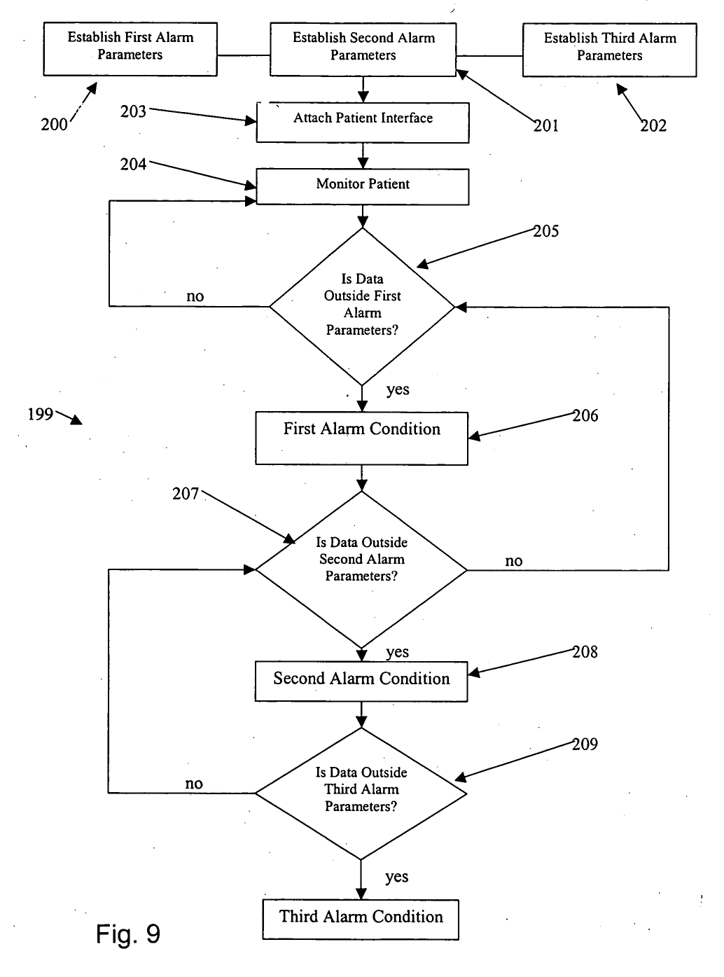

FIG. 9 illustrates one embodiment of method 199 for employing respiratory monitoring 11 having alarm responses. Step 200 of establishing first alarm parameters, comprises establishing predetermined parameters such as, for example, minimum pressure thresholds, that are programmed into controller 14. The predetermined parameters associated with step 200 comprise early warning parameters, where if a parameter or threshold is not met, it would indicate to user 13 that patient 18 needs to be carefully watched. Step 201 of establishing second alarm parameters, comprises establishing predetermined parameters associated with a moderately critical patient state. For example, thresholds established in step 201 may indicate a more critical patient situation than those established in step 200. Step 202 of establishing third alarm parameters comprises establishing predetermined parameters associated with a severely critical patient state. For example, thresholds established in step 202 may indicate a more critical patient situation than those established in step 201 or 200. It is in accordance with the present invention that a plurality of alarm responses be incorporated into method 199, where thresholds are established by evaluating any suitable patient parameter such as, for example, respiratory rate or respiratory pressure.

Method 199 further comprises step 203 of attaching the patient interface, consistent with step 101 (FIG. 8), and step 204 of monitoring the patient, consistent with step 102 (FIG. 8). While patient 18 is being monitored, method 199 queries whether data received by controller 14 is outside the established first alarm parameters, herein referred to as query 205. If the signals received by controller 14 fall inside the parameters established in step 200, method 199 will not activate first alarm condition 206 and will continue step 204 of monitoring the patient. If the signals received by controller 14 fall outside the parameters established in step 200, method 199 will proceed to step 206 of generating a first alarm condition.

The first alarm condition in step 206 comprises initiating a visual alarm via first series of LEDs 91 (FIG. 7) to user 13. The first alarm condition in step 206 may cause first series of LEDs 91 to flash repeatedly, revolve, or alert user 13 in any other suitable manner. In one embodiment of the present invention, first series of LEDs 91 is a color, e.g., white, distinguishable from inhalation LEDs 74, exhalation LEDs 67, second series of LEDs 92, and third series of LEDs 93. First alarm condition in step 206 may further initiate an auditory signal or alarm. In the event that respiratory monitoring 11 is integrated with drug delivery 19, as may be the case in sedation and analgesia systems or anesthesia delivery systems, the first alarm condition in step 206 may optionally initiate a step down or total deactivation of drug delivery rate associated with drug delivery 19.

The first alarm condition may generate a silent but visible alarm such as the white LED series lighting up to indicate that the anthropomorphic alarm algorithm has gone into a “hypervigilant” or attention mode. The alarm is silent so that it does not distract the user and because the conditions triggering the alarm are not serious enough to warrant distracting the user. However, to make sure that data is not being masked from the user, the white LEDs in series 91 light up as silent indicators. The first alarm condition may be triggered by the partial pressure of CO₂ averaged over e.g., 12 seconds, dropping below a threshold. In some instances, the first alarm condition may also be accompanied by a drug pause where administration of drugs is temporarily halted, especially if potent drugs are being administered.

Following the first alarm condition in step 206, method 199 will proceed to query whether data received by controller 14 is outside the parameters established in step 201, herein referred to as query 207. If the signals received by controller 14 fall within the parameters established in step 201, method 199 will return to query 205. If the signals received by controller 14 fall outside the parameters established in step 201, method 199 will proceed to the second alarm condition in step 208.

The second alarm condition in step 208 comprises, in one embodiment of the present invention, initiating a visual alarm via second series of LEDs 92 (FIG. 7) to user 13. The second alarm condition in step 208 may cause second series of LEDs 92 to flash repeatedly, revolve, or alert user 13 in any other suitable manner. In one embodiment of the present invention, second series of LEDs 92 is a color, e.g., orange, distinguishable from inhalation LEDs 74, exhalation LEDs 67, first series of LEDs 91, and third series of LEDs 93. The second alarm condition in step 208 may further initiate an auditory signal or alarm. In the event that respiratory monitoring 11 is integrated with drug delivery 19, as may be the case in sedation and analgesia systems and anesthesia delivery systems, the second alarm condition in step 208 may initiate a step down or total deactivation of drug delivery rate associated with drug delivery 19.

The second alarm condition may be synchronized with the messages displayed on the main user interface of a sedation and analgesia or anesthesia delivery system. Thus the orange LEDs in series 92 would light up in synchrony with an orange caution alarm on the main user interface of the sedation and analgesia system. A second alarm condition may be caused for example by a low respiratory rate.

Following the second alarm condition in step 208, method 199 will proceed to query whether data received by controller 14 is outside the parameters established in step 202, herein referred to as query 209. If the signals received by controller 14 fall within the parameters established in step 202, method 199 will return to query 207. If the signals received by controller 14 fall outside the parameters established in step 202, method 199 will proceed to the third alarm condition in step 210.

The third alarm condition in step 210 comprises, in one embodiment of the present invention, initiating a visual alarm via third series of LEDs 93 (FIG. 7) to user 13. The third alarm condition in step 210 may cause third series of LEDs 93 to flash repeatedly, revolve, or alert user 13 in any other suitable manner. In one embodiment of the present invention, third series of LEDs 93 is a color, e.g., red, distinguishable from inhalation LEDs 74, exhalation LEDs 67, first series of LEDs 91, and second series of LEDs 92. The third alarm condition in step 210 may further initiate an auditory signal or alarm. In the event that respiratory monitoring 11 is integrated with drug delivery 19, as may be the case in sedation and analgesia systems or anesthesia delivery systems, the third alarm condition in step 210 may initiate a step down or total deactivation of drug delivery rate associated with drug delivery 19. The third alarm condition may light the red LEDs in series 93 in synchrony with a red warning alarm on the main user interface of the sedation and analgesia system.

The present invention further comprises any suitable number of alarms or alarm condition steps, alerting user 13 in any suitable manner of a negative patient episode detected by controller 14, alarm condition steps that deactivate a plurality of critical patient peripherals such as, for example, a blood pressure cuff, reflective coverings positionable over ear mount 54, where light emitted from LEDs is magnified, and the use of method 100 in cooperation with method 199, and the use of respiratory monitoring 11 in the presence or absence of integrated oxygen delivery, analgesic delivery, and/or patient monitoring.

While exemplary embodiments of the invention have been shown and described herein, it will be obvious to those skilled in the art that such embodiments are provided by way of example only. Numerous insubstantial variations, changes, and substitutions will now be apparent to those skilled in the art without departing from the scope of the invention disclosed herein by the Applicants. Accordingly, it is intended that the invention be limited only by the spirit and scope of the claims as they will be allowed.

1. A respiratory monitoring system comprising:
a patient interface comprising a nasal cannula and a visual display, said nasal cannula comprising at least a first nasal capnography port and a first pressure sensor port and said visual display comprising indicators that are visible to a user while simultaneously observing a patient;
a respiratory monitor, comprising a sensor, wherein said respiratory monitor is adapted so as to be coupled to said patient interface and generate a signal reflecting at least one respiratory condition of the patient; and
an electronic controller interconnected with the respiratory monitor and the patient interface, wherein said visual display is modified based on the information contained in said signal.

2. The system of claim 1, further comprising a drug delivery device supplying one or more drugs to said patient, wherein said electronic controller receives said signal and manages said drug delivery device in response to said signal.

3. The system of claim 1, further comprising a user interface allowing a user to enter inputs, said inputs corresponding to thresholds for at least one respiratory parameter.

4. The system of claim 3, wherein said predetermined thresholds relate to inhalation or exhalation of said patient.

5. The system of claim 3, wherein pressure waveform analysis and segmentation is used to identify one of respiratory effort and effect based on said predetermined thresholds.

6. The system of claim 4, wherein alarm conditions are determined based on said one of respiratory effort and effect in relation to said predetermined thresholds.

7. The system of claim 4, wherein alarm conditions are determined based on other criteria in addition to said one of respiratory effort and effect in relation to said predetermined thresholds.

8. The system of claim 4, wherein said respiratory visual display comprises at least one series of light emitting diodes (LEDs) such that specific LEDs are associated with corresponding said one of respiratory effort and effect based on predetermined thresholds.

9. The system of claim 8, wherein said respiratory visual display is updated in real time.

10. The system of claim 8, wherein said LEDs are color coded to correspond to each type of said predetermined thresholds.

11. The system of claim 8, wherein said predetermined thresholds represent a gradual increase in magnitude of a corresponding parameter.

12. The system of claim 3, wherein said sensor includes at least one of a pressure sensor, humidistat, thermistor, and flow sensor.

13. The system of claim 1, further comprising an ear mount adapted for placement on at least one ear of a patient and from which said visual display can be mounted.

14. The system of claim 13, further comprising a support band coupled to said ear mount to provide stability to said ear mount and said visual display.

15. The system of claim 1, wherein said medical device is a sedation and analgesia system.

16. A method for implementing respiratory monitoring comprising:
attaching the patient interface, comprising fitting a patient with visual display and nasal cannula, wherein said visual display comprises a plurality of LED indicators indicating multiple levels of negative and positive pressure that are visible to a user while simultaneously observing a patient;
identifying a plurality of incremental thresholds for negative pressure readings and positive pressure readings;
integrating a respiratory monitoring device with said patient interface, wherein pressure variations caused by said patient's respiration pass to a sensor;
conducting a first query whether pressure sensed by said sensor is one of negative pressure and positive pressure;
if result of said first query is negative pressure, conducting a first negative pressure query to determine whether the negative pressure exceeds a first negative pressure threshold from said plurality of incremental thresholds for negative readings;
if result of said first query is positive pressure, conducting a first positive pressure query to determine whether the positive pressure exceeds a first positive pressure threshold from said plurality of incremental thresholds for positive readings;
if the result of said first negative pressure query exceeds said first negative pressure threshold or the result of said first positive pressure query exceeds said first positive pressure threshold, lighting a first pressure LED from said plurality of LED indicators corresponding to one of said first negative pressure threshold and said first positive pressure threshold; and
if the result of said first negative pressure query exceeds said first negative pressure threshold or the result of said first positive pressure query exceeds said first positive pressure threshold, conducting at least one additional negative pressure query or positive pressure query.

17. The method of claim 16, wherein said step of integrating further comprises providing a plurality of sensors in cooperation with said pressure sensor.

18. The method of claim 16, wherein said negative pressure reading or said positive pressure reading of sufficient magnitude to cross multiple of said plurality of incremental thresholds results in simultaneous illumination of each LED corresponding to each of said crossed thresholds.

19. The method of claim 18, wherein pulse width modulation (PWM) of an electrical supply is delivered to an LED array such that, as a greater number of said plurality of incremental thresholds are crossed, the pulse width of said electrical supply is increased, resulting in brighter light intensity of the LEDs.

20. A method for employing respiratory monitoring having alarm responses, comprising:
establishing first alarm parameters comprising minimum pressure thresholds that are programmed into a;
establishing second alarm parameters associated with a moderately critical patient state;
establishing third alarm parameters associated with a severely critical patient state;
attaching a patient interface, comprising fitting a patient with visual display and nasal cannula, wherein said visual display comprises a plurality of LED indicators;
monitoring the patient, wherein said monitoring produces data regarding said patient;
querying whether said data is outside said first alarm parameters;
if said data falls outside said first alarm parameters, generating a first alarm condition;
querying whether said data is outside said second alarm parameters;
if said data falls outside said second alarm parameters, generating a second alarm condition;
querying whether said data is outside said third alarm parameters; and
if said data falls outside said third alarm parameters, generating a third alarm condition.

21. The method of claim 20, wherein said visual display comprises a first series of LEDs, a second series of LEDs, a third series of LEDs, an inhalation LED, and an exhalation LED.

22. The method of claim 21, wherein said first, second and third series of LEDs is a color distinguishable from each other and from said inhalation LED and said exhalation LED.

23. The method of claim 22, wherein said first alarm condition comprises initiating a visual alarm via said first series of LEDs, said second alarm condition comprises initiating a visual alarm via said second series of LEDs, and said third alarm condition comprises initiating a visual alarm via said third series of LEDs.

24. The method of claim 23, wherein at least one of said first, second and third alarm condition further comprises initiating an auditory signal or alarm.

25. The method of claim 20, wherein at least one of said first, second and third alarm condition comprises initiating a step down of drug delivery rate associated with a drug delivery.

26. The method of claim 20, wherein at least one of said first, second and third alarm condition comprises deactivating one or more patient peripherals.

27. The method of claim 20, wherein at least one of said second and third alarm condition is based on a patient's respiratory rate.
